(19) United States
(12) Patent Application Publication
(10) Pub. No.: US 2025/0144419 A1
(43) Pub. Date: May 8, 2025

(54) WEARABLE DEVICE FOR ALLEVIATING LOWER LIMB ISCHEMIA AND CONTROL METHOD THEREFOR

(71) Applicants: The Eighth Affiliated Hospital of Sun Yat-Sen University, Shenzhen (CN); Shenzhen Weihang Medical Devices Co., Ltd, Shenzhen (CN)

(72) Inventors: Jianhang Du, Shenzhen (CN); Hui Huang, Shenzhen (CN); Yuyu Lin, Shenzhen (CN)

(21) Appl. No.: 19/012,929

(22) Filed: Jan. 8, 2025

Related U.S. Application Data
(63) Continuation of application No. PCT/CN2022/126779, filed on Oct. 21, 2022.

(30) Foreign Application Priority Data
Jul. 20, 2022 (CN) 202210874545.X

(51) Int. Cl. A61N 1/36; A61N 1/02; A61N 1/04

(52) U.S. Cl. CPC A61N 1/36034; A61N 1/025; A61N 1/0452; A61N 1/36003; A61N 1/36031

(57) ABSTRACT

Disclosed in the present disclosure are a wearable device for alleviating lower limb ischemia, and a control method therefor. The device comprises a main control unit, an electrocardiosignal monitoring unit, a first electrical pulse assembly and a second electrical pulse assembly. The first electrical pulse assembly and the second electrical pulse assembly are each composed of an intermediate-frequency pulse stimulation electrode having a pulse frequency ranging from 1 Kz to 100 Kz and a low-frequency pulse stimulation electrode having a pulse frequency ranging from 50 Hz to 300 Hz. In the control method, a control algorithm is set on the basis of an electrocardiosignal, such that a first pulse assembly and a second pulse assembly output electronic pulses at specific modulation waves and according to a preset instruction, so as to generate a hammering effect, thereby achieving the effects of improving the blood flow velocity at the lower limbs.

### CROSS-REFERENCE TO RELATED APPLICATIONS

The present application is a continuation application of PCT application No. PCT/CN2022/126779 filed on Oct. 21, 2022, which claims the benefit of Chinese Patent Application No. 202210874545.X filed on Jul. 20, 2022. The contents of all of the aforementioned applications are incorporated by reference herein in their entirety.

### FIELD OF THE INVENTION

The present disclosure belongs to the technical field of medical devices, relates to a physiotherapy apparatus and a control method, and particularly relates to a wearable device for alleviating lower limb ischemia and a control method therefor.

### BACKGROUND OF THE INVENTION

Peripheral arterial disease (PAD) of lower limbs is one of the most common vascular diseases, affecting more than 200 million people worldwide. Patients with the PAD of the lower limbs are often accompanied by the problem of limb ischemia, which is manifested as intractable foot pain at rest, and tissue necrosis in severe cases, which is mainly caused by thromboarteritis arteritis, vascular endothelial dysfunction of lower limbs and atherosclerotic lesions. Common diseases resulting therefrom include diabetic feet, arteriosclerotic occlusive disease, thromboangiitis obliterans, etc., and an end stage of the PAD is accompanied by severe limb ischemia, resulting in impaired quality of life, serious complications and even death. Survey data have shown that for conditions of severe lower limb ischemia (CLI) in the end stage of the peripheral arterial disease of the lower limbs, the amputation rate of patients in one year is as high as 30%, the mortality rate is as high as 25%, and the long-term mortality rate is higher than that of patients with symptomatic coronary heart disease.

To alleviate the PAD, controlling inflammation and improving lower limb hemodynamics are two key means, especially the latter is more important, which not only runs through a development stage, an end stage and a rehabilitation stage of the disease, but also affects the control of inflammation. Currently, there are many treatment methods for lower limb ischemic diseases, such as lifestyle adjustment, drug treatment, interventional therapy, surgical treatment, etc., but it is difficult to achieve ideal results. Improvement of the lifestyle is mainly achieved by exercise, smoking cessation, etc., this method is more suitable for mild lower limb ischemic disease, and the improvement effect on severe patients is not obvious; the drug treatment uses drugs for antithrombotic and lipid-lowering purposes, and its effect on improving lower limb hemodynamics is also very limited; and traditional surgery has a high residual rate of thrombus, and trauma of the surgery increases the recurrence rate of thrombus and the risk of incision infection, and additionally, the surgery has the disadvantages of high demands on patients, poor vascular intervention, and the like.

In recent years, in addition to the above methods, some new physical therapies and mechanical preventive therapies have been developed, mainly employing intermittent pneumatic compression (IPC), graduated compression stockings (GCSs), venous foot pumps (VFPs) and enhanced external counterpulsation (EECP), etc. The compression therapy employing GCSs and VFPs is mainly used for the treatment of lower limb venous diseases, such as chronic venous insufficiency diseases such as lower leg edema and varicose veins, and prevention of deep vein thrombosis, with little effects on lower limb ischemic diseases. The IPC is considered to have a certain effect on improving the inflow of lower leg artery, which is conducive to the development of collateral circulation and the improvement of intermittent claudication. However, more clinical evidence-based evidence is still needed to verify its efficacy and safety. The IPC has a very limited effect on improving the blood flow velocity of blood vessels in the lower limbs due to its lack of cooperation with the cardiac motion process. Moreover, because the IPC employs an airbag that tightly wraps the foot, lower leg and thigh, if a patient has lower limb infection, the airbag structure will have a negative impact on an infected area, increasing the risk of infection deterioration. A main function of the EECP is to improve the blood perfusion of important organs in the upper body, the effects of improving the blood flow velocity of the lower limbs are currently questionable, and there is even a possibility of reducing the inflow of the blood flow in the lower limbs during a cardiac cycle, and the high air pressure used by the EECP also has the risk of causing problems such as skin abrasions, contusions, and muscular soreness in the lower limbs, especially in the case of lesions or infectious complications of the lower limbs. In addition, for both methods of IPC and EECP, which require high-pressure gas generating and controlling components, a device is high in size, weight and cost, is complicated in operation, needs to be operated by professional medical staff in order to prevent accidents, has a small application range, and is not suitable for use by non-medical institutions, particularly ordinary households.

Taken together, the three compression therapies described above all have relatively limited improvements in the hemodynamic effects of the lower limbs, and in addition, the potential serious risks of these mechanical stimulation-based compression therapies also limit their clinical promotion and application.

In view of this, it is necessary to further improve the existing device for alleviating lower limb ischemia and a control method therefor to improve the improvement effect, reduce the risk and cost, and expand the application range thereof.

### SUMMARY OF THE INVENTION

To this end, the technical problems to be solved by the present disclosure are that the conventional means for alleviating lower limb ischemia has poor effects and risks, high cost, and difficult operation. Therefore, a wearable state for improving the problem of lower limb ischemia which has a good improvement effect, low risk, good portability, and easy operation and a control method therefor are proposed.

To solve the above technical problems, the technical solutions of the present disclosure are as follows:
according to a first aspect of the present disclosure, provided is a wearable device for alleviating lower limb ischemia, including:
a main control unit,
an electrocardiosignal monitoring unit configured to be worn on a chest of a human body, and being in signal connection with the main control unit;
a first electrical pulse assembly configured to be worn on a thigh of the human body, and being in signal connection with the main control unit, wherein the first electrical pulse assembly includes at least two groups of first pulse stimulation electrodes, each group of the first pulse stimulation electrodes includes at least three first pulse stimulation electrodes, the at least two groups of the first pulse stimulation electrodes are respectively disposed corresponding to an outer thigh and an inner thigh, and one ends, away from the electrocardiosignal monitoring unit, of the first pulse stimulation electrodes are provided with at least one group of third pulse stimulation electrodes; and
a second electrical pulse assembly configured to be worn on a lower leg of the human body, and being in signal connection with the main control unit, wherein the second electrical pulse assembly includes at least two groups of second pulse stimulation electrodes, each group of the second pulse stimulation electrodes includes at least three second pulse stimulation electrodes, the at least two groups of the second pulse stimulation electrodes are respectively disposed corresponding to a lateral side and a medial side of the lower leg, and one ends, away from the electrocardiosignal monitoring unit, of the second pulse stimulation electrodes are provided with at least one group of fourth pulse stimulation electrodes, wherein
the first pulse stimulation electrodes and the second pulse stimulation electrodes have a pulse frequency of 1-100 KHz, and the third pulse stimulation electrodes and the fourth pulse stimulation electrodes have a pulse frequency of 50-300 Hz.

Preferably, the first electrical pulse assembly further includes a first wearing portion, the first wearing portion includes a first wearing portion body, the first wearing portion body is connected with at least one first adjustable connecting part, a wearing space is enclosed by the first wearing portion body and the first adjustable connecting part, and the first pulse stimulation electrodes and the third pulse stimulation electrodes are attached to an inner wall of the first wearing portion body; and the first wearing portion body is also connected with a first temperature control module.

Preferably, the second electrical pulse assembly further includes a second wearing portion, the second wearing portion includes a second wearing portion body, the second wearing portion body is connected with at least one second adjustable connecting part, a wearing space is enclosed by the second wearing portion body and the second adjustable connecting part, and the second pulse stimulation electrodes and the fourth pulse stimulation electrodes are attached to an inner wall of the second wearing portion body; and the second wearing portion body is also connected with a second temperature control module.

Preferably, the main control unit includes an adjustable wristband and a main controller connected to the adjustable wristband, and the main controller includes a microprocessor, and a communication module, a data analysis and processing module, a data storage module, a display module and a control module which are in signal connection with the microprocessor.

Preferably, the electrocardiosignal monitoring unit includes an adjustable chest strap and an electrocardiosignal acquisition module connected to the adjustable chest strap, the electrocardiosignal acquisition module being wirelessly connected with the communication module.

Preferably, the first adjustable connecting part includes first hinged connectors connected to one end of the first wearing portion body, and first adjustable elastic bands connected to the other end of the first wearing portion body; and the second adjustable connecting part includes second hinged connectors connected to one end of the second wearing portion body, and second adjustable elastic bands connected to the other end of the second wearing portion body.

According to a second aspect of the present disclosure, provided is a control method for the wearable device for alleviating lower limb ischemia, including the steps of:
obtaining human body signals, the human body signals including at least electrocardiosignals;
processing the human body signals, and generating control signals according to a processing result including R-wave signals, T-wave signals, or P-wave signals in the obtained electrocardiosignals; and
controlling the first electrical pulse assembly and the second electrical pulse assembly to output pulse modulated waves based on the control signals to alternately perform positive stimulation and negative stimulation.

Preferably, controlling the first electrical pulse assembly and the second electrical pulse assembly to output pulse modulated waves based on the control signals for positive stimulation includes:
controlling the second pulse stimulation electrodes to sequentially output pulse modulated waves in a direction from a distal end of a lower leg to a proximal end of the lower leg; and
controlling the first pulse stimulation electrodes to sequentially output pulse modulated waves in a direction from a distal end of a thigh to a proximal end of the thigh after a preset time interval.

Preferably, controlling the first electrical pulse assembly and the second electrical pulse assembly to output pulse modulated waves based on the control signals for negative stimulation includes:
controlling the first pulse stimulation electrodes to sequentially output pulse modulated waves in a direction from the proximal end of the thigh to the distal end of the thigh;
controlling the second pulse stimulation electrodes to sequentially output pulse modulated waves in a direction from the proximal end of the lower leg to the distal end of the lower leg; and
until T-waves are monitored, controlling the first pulse stimulation electrodes and the second pulse stimulation electrodes to stop working.

Preferably, the negative stimulation further includes a step of controlling the third pulse stimulation electrodes and the fourth pulse stimulation electrodes to output pulse modulated waves; and
the method further includes the steps of controlling the third pulse stimulation electrodes and the fourth pulse stimulation electrodes to output pulse modulated waves, and controlling the first electrical pulse assembly and the second electrical pulse assembly to be heated to 35-45° C. before controlling the first electrical pulse assembly and the second electrical pulse assembly to output pulse modulated waves based on the control signals to alternately perform positive stimulation and negative stimulation.

Compared with the prior art, the above technical solutions of the present disclosure have the following advantages:
(1) the wearable device for alleviating lower limb ischemia provided by the present disclosure includes the main control unit, the electrocardiosignal monitoring unit, the first electrical pulse assembly, and the second electrical pulse assembly, both the first electric pulse assembly and the second electric pulse assembly consist of intermediate-frequency pulse stimulation electrodes with a pulse frequency of 1-100 Kz and low-frequency pulse stimulation electrodes with a pulse frequency of 50-300 Hz, thereby intervening in the movement of blood vessels in the lower limbs by means of intermediate-frequency electronic pulses as a main power source in the thigh and the lower leg, respectively, and supplementing with low-frequency electronic pulses to reduce the impedance of tiny blood vessels at a terminal end, and the device is a non-invasive, low-risk, hemodynamically defined, wireless communication-connected, more miniaturized, portable, and low-cost wearable device, and can achieve more precise multi-level intervention without the need for professional medical staff to operate, effectively increasing the blood flow velocity of the lower limbs and achieving the effect of increasing the perfusion of the blood flow flowing to distal ends of the lower limbs. Compared with conventional high air pressure driven compression devices, the wearable device also has the advantages of reduced size, weight, and cost, and can avoid risks such as skin damage caused by high pressure.
(2) In the control method for the wearable device for alleviating lower limb ischemia provided by the present disclosure, a control algorithm is set based on electrocardiosignals, so that the first electrical pulse assembly and the second electrical pulse assembly output electronic pulses at specific modulation waves according to a preset instruction to produce a hammering effect, prompting lower limb muscles and blood vessels to generate regular deformation and movement, which is in cooperation with the human heart movement, thereby achieving the purposes of improving the hemodynamic environment of the lower limbs, effectively increasing the blood flow velocity of the lower limbs, and increasing the perfusion of the blood flow flowing to the distal ends of the lower limbs. The control method has high accuracy and can still control the pulse stimulation electrodes to work effectively even in the case of unstable and poor-quality electrocardiosignals.

### BRIEF DESCRIPTION OF DRAWINGS

In order to make the contents of the present disclosure more clearly understood, the present disclosure will be described in further detail below with reference to specific embodiments of the present disclosure and in conjunction with the accompanying drawings, wherein:

FIG. 1 is a schematic diagram of a wearing state of a wearable device for alleviating lower limb ischemia provided in Embodiment 1 of the present disclosure;

FIG. 2 is a structural schematic diagram of a main control unit in the wearable device provided in Embodiment 1 of the present disclosure;

FIG. 3 is an exploded schematic view of the main control unit in the wearable device provided in Embodiment 1 of the present disclosure;

FIG. 4 is a structural schematic diagram of a main controller in the wearable device provided in Embodiment 1 of the present disclosure;

FIG. 5 is a structural schematic diagram of a first electrical pulse assembly in the wearable device provided in Embodiment 1 of the present disclosure;

FIG. 6 is a structural schematic diagram of a second electrical pulse assembly in the wearable device provided in Embodiment 1 of the present disclosure;

FIG. 7 is a principle block diagram of the wearable device provided in Embodiment 1 of the present disclosure;

FIG. 8 is a flow chart of a control method provided in Embodiment 2 of the present disclosure;

FIG. 9 is a diagram showing electrocardiosignals obtained in the control method provided in Embodiment 2 of the present disclosure;

FIG. 10 is a schematic diagram of pulse output of positive stimulation in the control method provided in Embodiment 2 of the present disclosure; and

FIG. 11 is a schematic diagram of pulse output of negative stimulation in the control method provided in Embodiment 2 of the present disclosure.

The reference numerals in the figures are indicated as: 1—main control unit; 101—adjustable wristband; 102—main controller; 1021—housing; 1022—touch screen; 1023—control button; 1024—function button; 1025—sync button; 1026—data interface; 1027—charging interface; 2—electrocardiosignal monitoring unit; 201—adjustable chest strap; 202—electrocardiosignal acquisition module; 3—first electrical pulse assembly; 301—first pulse stimulation electrode; 302—third pulse stimulation electrode; 303—first wearing portion body; 304—first hinged connector; 305—first adjustable elastic band; 306—first light display module; 307—first pulse charging interface; 308—first pulse display module; 4—second electrical pulse assembly; 401—second pulse stimulation electrode; 402—fourth pulse stimulation electrode; 403—second wearing portion body; 404—second hinged connector; 405—second adjustable elastic band; 406—second light display module; and 407—second pulse display module.

### DETAILED DESCRIPTION OF THE INVENTION

In order to make the objects, technical solutions and advantages of the embodiments of the present disclosure more clear, the technical solutions in the embodiments of the present disclosure will be clearly and completely described below in conjunction with the accompanying drawings in the embodiments of the present disclosure, and obviously, the described embodiments are some, but not all, of the embodiments of the present disclosure. The components in the embodiments of the present disclosure generally described and illustrated in the drawings herein can be arranged and designed in a variety of different configurations.

Thus, the following detailed description of the embodiments of the present disclosure provided in the drawings is not intended to limit the scope of the present disclosure, but is merely to represent selected embodiments of the present disclosure. Based on the embodiments in the present disclosure, all other embodiments obtained by those of ordinary skill in the art without making inventive steps belong to the scope of protection of the present disclosure.

In the description of the present disclosure, it needs to be understood that the orientation or positional relationship indicated by the terms “upper”, “lower” and the like is based on the orientation or positional relationship shown in the drawings, or the orientation or positional relationship in which the product of the present disclosure is conventionally placed in use, or the orientation or positional relationship commonly understood by those skilled in the art, only for ease of describing the present disclosure and for simplicity of description, and not indicating or implying that the device or element referred must have a specific orientation, and be constructed and operated in a specific orientation, and therefore cannot be construed as limiting the present disclosure.

The terms “first”, “second”, etc. in the present disclosure are merely used for distinction in the description and have no special meaning.

In the description of the present disclosure, it should also be noted that unless otherwise expressly specified and defined, the terms “disposed” and “mounted” should be understood in a broad sense, e.g., they may be fixedly connected, removably connected, or integrally connected; they may be directly connected or may be indirectly connected through an intermediate medium. The specific meaning of the above terms in the present disclosure will be understood by those of ordinary skill in the art according to specific situations.

### Embodiment 1

This embodiment provides a wearable device for alleviating lower limb ischemia, which is used for intervening in the blood flow velocity and flow direction, thereby alleviating the problem of lower limb ischemia, and can be applied to the technical field of treatment of lower limb ischemic disease.

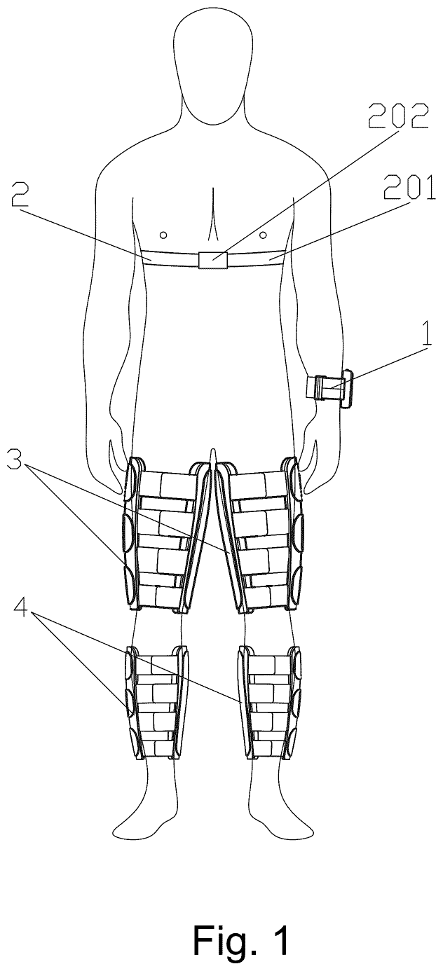

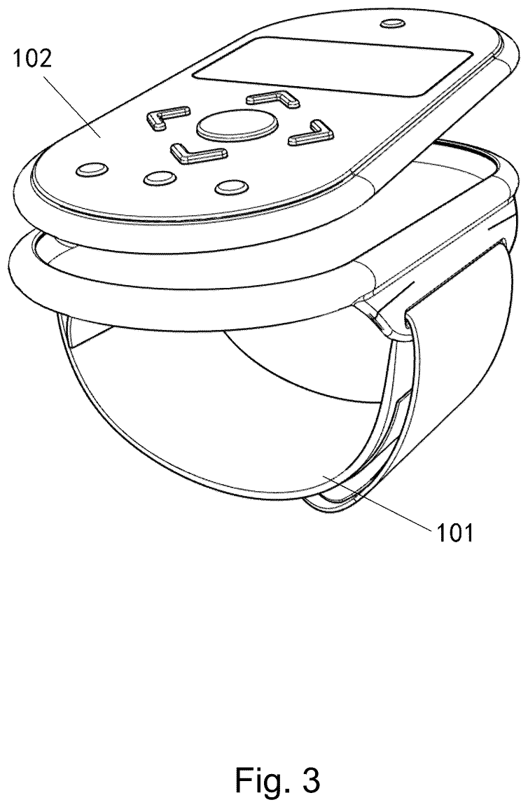

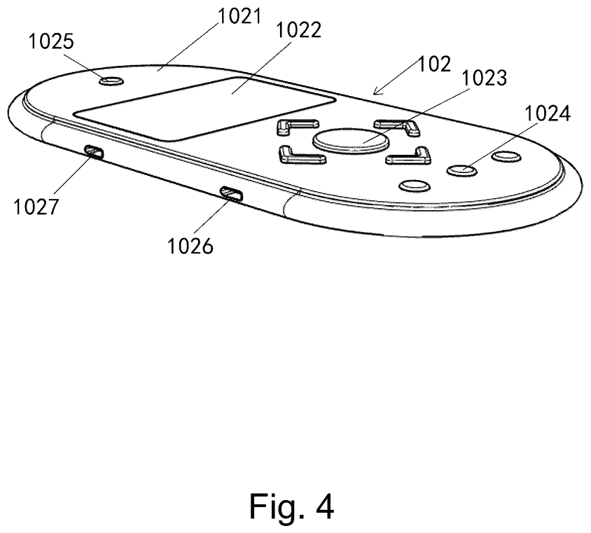

Referring to FIGS. 1 to 6, the wearable device includes a main control unit 1, in this embodiment, the main control unit 1 is preferably a wrist-worn main control device for being worn on a wrist of a human body to control other components, and the main control unit 1 is in signal connection with an electrocardiosignal monitoring unit 2 which preferably has a chest strap type structure, and can be worn around a chest of the human body to monitor electrocardiosignals of the human body and transmit the electrocardiosignals to the main control unit 1.

The main control unit 1 is also in signal connection with a first electrical pulse assembly 3, and the first electrical pulse assembly 3 can be worn on a thigh of the human body, and is configured to output electrical pulses to the thigh of the human body. In particular, the first electrical pulse assembly 3 includes at least two groups of first pulse stimulation electrodes 301, the two groups of the first pulse stimulation electrodes 301 are respectively disposed corresponding to an inner thigh and an outer thigh, and each group of the first pulse stimulation electrodes 301 includes at least three first pulse stimulation electrodes 301, in this embodiment, each group of the first pulse stimulation electrodes 301 includes three first pulse stimulation electrodes 301 which are sequentially arranged at intervals along the length direction of the thigh, so that the first pulse stimulation electrodes 301 can stimulate blood vessels in different muscle parts of the thigh, in this embodiment, the two groups of the first pulse stimulation electrodes 301 are used to be closely adhered to the vastus lateralis muscle and the vastus medialis muscle, respectively, to stimulate blood vessels on both the inner thigh and the outer thigh, respectively, and the first pulse stimulation electrodes 301 have a pulse frequency of 1-100 KHz, a pulse width of 200-400 μs, a maximum output amplitude effective value of 25 V or less (50 mA), and a maximum output energy of a single pulse being 300 mJ or less, and intermediate-frequency pulses. One ends, away from the electrocardiosignal monitoring unit 2, of the first pulse stimulation electrodes 301 are further provided with third pulse stimulation electrodes 302, and ends, close to the heart, of the thigh and a lower leg are defined as proximal ends, and ends, away from the heart, of the thigh and the lower leg are defined as distal ends according to the distance from the heart of the human body, i.e., the third pulse stimulation electrodes 302 are located at the distal end of the thigh. In this embodiment, one group of the third pulse stimulation electrodes 302 is provided, and one group of the third pulse stimulation electrodes 302 includes two pulse stimulation electrodes corresponding to the outer thigh and the inner thigh, respectively, and the third pulse stimulation electrodes 302 have a pulse frequency of 50-300 Hz, a pulse width of 100-300 μs, a maximum output energy of 250 mJ or less, and a single pulse power at a maximum output amplitude of 6 μC or more; a maximum output amplitude effective value of 25 V or less (50 mA), and low-frequency pulses. Of course, as an alternative embodiment, other numbers of each group of the first pulse stimulation electrodes 301 and the third pulse stimulation electrodes 302 may be used, as long as they function to stimulate blood vessels at different thigh muscle locations, without limitation.

The main control unit 1 is also in signal connection with a second electrical pulse assembly 4 which can be worn on the lower leg of the human body, the second electrical pulse assembly 4 includes at least two groups of second pulse stimulation electrodes 401, each group of the second pulse stimulation electrodes 401 includes at least three second pulse stimulation electrodes 401, in this embodiment, the second electrical pulse assembly 4 includes two groups of the second pulse stimulation electrodes 401, with three second pulse stimulation electrodes 401 in each group, each group of the second pulse stimulation electrodes 401 is sequentially arranged at intervals in a direction from the proximal end to the distal end of the lower leg, wherein the two groups of the second pulse stimulation electrodes 401 correspond to a lateral side and a posterior medial side of the lower leg, respectively, the second pulse stimulation electrodes 401 corresponding to the lateral side of the lower leg may be closely attached to the peroneus muscle to the tibialis anterior muscle, the second pulse stimulation electrodes 401 corresponding to the posterior medial side of the lower leg may be closely attached to the soleus muscle to the gastrocnemius muscle, and the second pulse stimulation electrodes 401 have a pulse frequency of 1-100 KHz, a pulse width of 200-400 μs, a maximum output amplitude effective value of 25 V or less (50 mA), a maximum output energy of a single pulse being 300 mJ or less, and intermediate-frequency pulses. One ends, away from the electrocardiosignal monitoring unit 2, pf the two groups of the second pulse stimulating electrodes 401 are provided with at least one group of fourth pulse stimulation electrodes 402, i.e., the fourth pulse stimulation electrodes 402 are disposed corresponding to the distal end of the lower leg. In this embodiment, one group of the fourth pulse stimulation electrodes 402 is provided, and one group of the fourth pulse stimulation electrodes 402 includes two pulse stimulation electrodes corresponding to the lateral side and the posterior medial side of the lower leg, respectively, and the fourth pulse stimulation electrodes 402 have a pulse frequency of 50-300 Hz, a pulse width of 100-300 μs, a maximum output energy of 250 mJ or less, and a single pulse power at a maximum output amplitude of 6 μC or more; a maximum output amplitude effective value of 25 V or less (50 mA), and low-frequency pulses. Of course, as an alternative embodiment, other numbers of each group of the second pulse stimulation electrodes 401 and the fourth pulse stimulation electrodes 402 may be used, as long as they function to stimulate blood vessels at different lower leg muscle locations, without limitation.

In the wearable device provided by this embodiment, the first electrical pulse assembly 3 and the second electrical pulse assembly 4 are arranged according to the position and orientation of the lower limb arteries (peroneal artery, tibial artery, popliteal artery, femoral artery, and iliac artery) and veins (peroneal vein, posterior tibial vein, popliteal vein, and saphenous vein), the first pulse stimulation electrodes 301 in the first electrical pulse assembly 3, and the second pulse stimulation electrodes 401 in the second electrical pulse assembly 4 function to output intermediate-frequency pulses, and the intermediate-frequency pulses are modulated and output as square waves, which can produce a hammering effect and an equivalent pressure of 10 Kpa level, thus effectively inducing deformation and movement of lower leg and thigh muscles and blood vessels, ultimately achieving the goal of intervening and regulating lower limb blood circulation. In addition, the third pulse stimulation electrodes 302 and the fourth pulse stimulation electrodes 402 function to output low-frequency pulses to limb muscles, dilate blood vessels and reduce the impedance of limb blood vessels, so that intermediate-frequency electrical pulses and low-frequency electrical pulses can be used to stimulate the corresponding arteries and veins of the thigh and the lower leg.

The wearable device for alleviating lower limb ischemia provided by this embodiment intervenes in the movement of blood vessels in the lower limbs by means of intermediate-frequency electronic pulses as main power source in the thigh and the lower leg, respectively, supplemented with low-frequency electronic pulses to reduce the impedance of tiny blood vessels at a terminal end, is a non-invasive, low-risk, hemodynamically defined, wireless communication-connected, more miniaturized, portable, and low-cost wearable device, and can achieve more precise multi-level intervention without the need for professional medical staff to operate, effectively increasing the blood flow velocity of the lower limbs, and achieving the effect of increasing the perfusion of the blood flow flowing to distal ends of the lower limbs, and can be widely applied to primary medical institutions or families, and has great value and application prospects as a non-drug, non-surgical lower limb blood flow promotion and rehabilitation physiotherapy device.

Compared with conventional high air pressure driven compression devices (such as IPC, GCS, VFPs, and EEPC), the wearable device with a combination of medium and low-frequency electronic pulses of a specific frequency band as a power source has the advantages of energy concentration, fast response speed, small device size, light weight, and low cost, can also improve the accuracy of intervention, and can avoid risks such as skin damage caused by high pressure. In addition, the arrangement of the first electrical pulse assembly 3 and the second electrical pulse assembly 4 is determined based on the orientation characteristics of arterial and venous vessels in the thigh and the lower leg, significantly improving the efficiency of inducing vascular deformation.

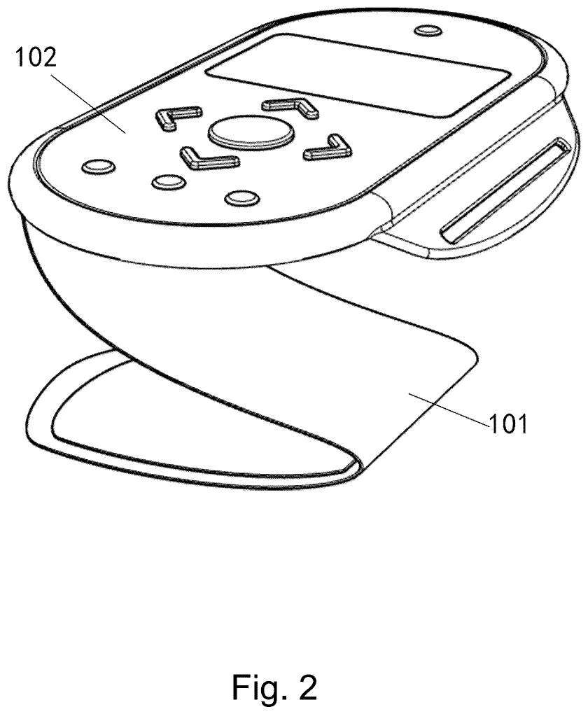

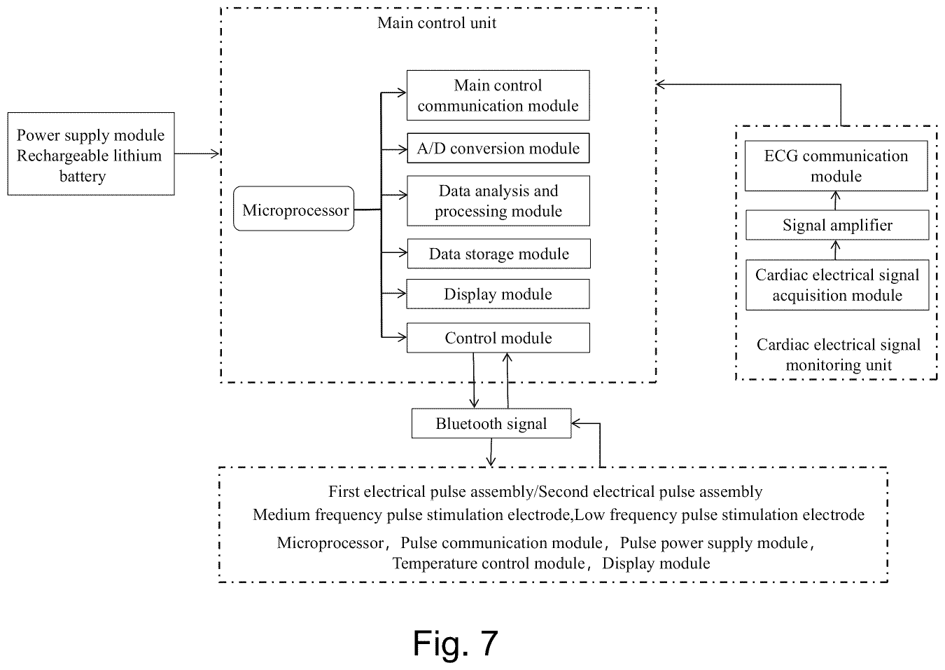

Specifically, referring to FIGS. 2 to 4, the main control unit 1 is a wrist-worn main control unit, including an adjustable wristband 101 configured to be worn on a left wrist of a human body, wherein the adjustable wristband 101 may employ a wristband with hook and loop fasteners, and is provided with a mounting portion, when the main control unit 1 is worn on a wrist of the human body, two ends of the adjustable wristband 101 are attached to each other by the hook and loop fasteners matched with each other, a main controller 102 is detachably connected to the mounting portion of the adjustable wristband 101, the main controller 102 includes a housing 1021, and functional modules (as shown in FIG. 7) disposed inside the housing 1021, and the functional modules include: a microprocessor, the microprocessor is in signal connection with a main control communication module, in this embodiment, the main control communication module is preferably a Bluetooth communication module, the microprocessor is also in signal connection with a data analysis processing module, a data storage module, a display module, a control module, and an A/D conversion module, the control module uses a micro control unit AVR MCU chip (e.g., ATmega8 chips from ATMEL, Inc.), which has multiple 16-bit and 8-bit timers, and can achieve multiple, sequential, distributed intervention modes, and can accurately calculate the time node and time interval of electric pulse generation and issue instructions to a waveform generator (CPLD) of the pulse assembly.

The A/D conversion module is configured to perform A/D conversion on electrocardiosignals obtained by the electrocardiosignal monitoring unit 2. To supply power to the main control unit 1, the microprocessor is also connected to a power module, preferably a rechargeable lithium battery.

A touch screen 1022 is connected to one surface of the housing 1021, the touch screen 1022 is connected to the display module, and is also connected with a control button 1023, function keys 1024 and a sync button 1025, and the above buttons are used to implement different control functions, and a data interface 1026 for data transmission and a charging interface 1027 for being connected with an external power supply are disposed at one side of the housing 1021.

The electrocardiosignal monitoring unit 2 has a chest strap type structure, can be worn on a chest of the human body, and includes an adjustable chest strap 201 and an electrocardiosignal acquisition module 202 connected to the adjustable chest strap 201, wherein the adjustable chest strap 201 is an elastic strap or has a strap structure of which both ends are connected by fasteners or hook and loop fasteners, as shown in FIG. 7, the electrocardiosignal acquisition module 202 includes an ECG communication module, which is preferably a low-power Internet of Things BLE4.0/5.0 Bluetooth communication module which is in signal connection with the main control communication module, and also includes a signal amplifying module and a signal collecting electrode which are connected to each other, and the signal collecting electrode uses a fabric electrode, and is configured to obtain electrocardiosignals of the human body, and the obtained electrocardiosignals are amplified and analog-to-digital converted by the signal amplifying module and transmitted to the main control unit 1 through the ECG communication module. The signal amplifying module includes an amplification circuit, in this embodiment, the signal amplification circuit employs a precision instrument amplifier (such as AD62* Series from AD Corporation), the amplification circuit is further connected with a high-pass and low-pass filter/notch circuit and an analog-to-digital (A/D) conversion circuit, where the analog-to-digital conversion circuit employs a high-speed low-power 16-bit analog-to-digital (A/D) converter (e.g., AD7705 from AD Corporation, TLC548/549 from TI Corporation, etc.), and the signal collecting electrode is connected to a DSP control chip which serves as a core device of a data acquisition and transmission control hardware processing circuit, and may employ a TMS320LF2407 chip from TI Corporation.

To supply power to the electrocardiosignal monitoring unit 2, a rechargeable lithium battery is connected to the electrocardiosignal acquisition module 202. The chest strap type electrocardiosignal monitoring unit 2 provided by this embodiment identifies and extracts ECG R, S, T and P waves based on a DSP chip, and transmits the ECG R, S, T and P waves to the main control unit 1 after analog-to-digital conversion, the above electrocardiosignal monitoring unit 2 using wireless transmission is simple in structure, easy to wear, and simple to operate and better in comfort compared with the conventional extracorporeal counterpulsation apparatus using wet ECG electrode patches and complicated wire connection.

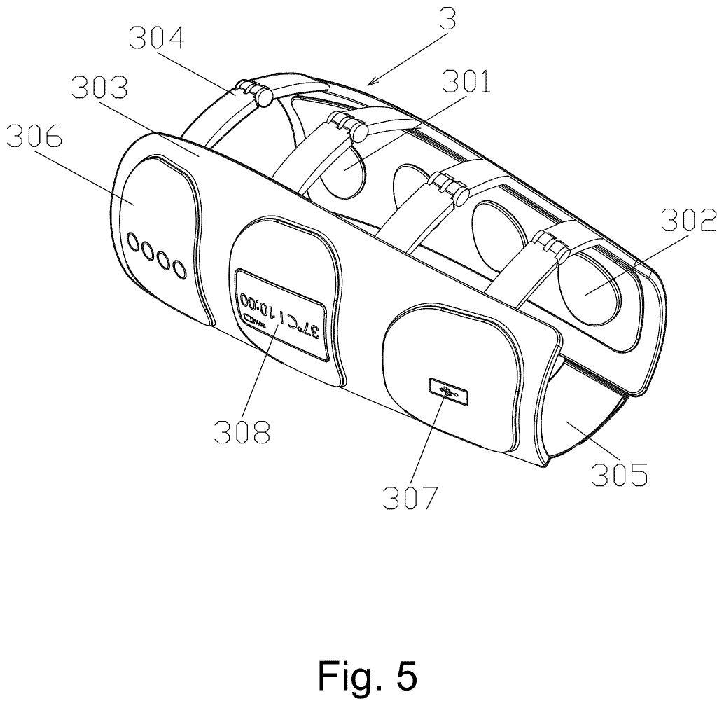

As shown in FIG. 5, the first electrical pulse assembly 3 provided in this embodiment includes a first wearing portion, the first wearing portion specifically includes a first wearing portion body 303, the first wearing portion body 303 is made of plastic, the first wearing portion body 303 includes two sheet-like structures having a circular arc-shaped curved surface which are oppositely disposed, the two sheet-like structures are adapted to be adhered to the inner thigh and the outer thigh of the human body, the first wearing portion body 303 is connected with at least one first adjustable connecting part, a wearing space is enclosed by the first adjustable connecting part and the first wearing portion body 303, and by adjusting the first adjustable connecting part, an inner wall surface of the first wearing portion body 303 is closely adhered to the thigh, wherein the two groups of the first pulse stimulation electrodes 301 and the two third pulse stimulation electrodes 302 are respectively attached to an inner side wall of the first wearing portion body 303, and arranged at intervals along the length direction of the first wearing portion body 303, and the first pulse stimulation electrodes 301 and the third pulse stimulation electrodes 302 are each disc-shaped electrodes. The first adjustable connecting part includes first hinged connectors 304 connected to one end of the first wearing portion body 301, and first adjustable elastic bands 305 connected to the other end of the first wearing portion body 303, specifically, the first hinged connectors 304 are connected to side edges of the two sheet-like structures, respectively, connecting the two sheet-like structures, and the other ends of the two sheet-like structures are separately connected with the first adjustable elastic bands 305, and the first adjustable elastic bands 305 can be further adjusted for tightness by a hook and loop fastener structure.

Further, the first wearing portion body 303 is also connected with a first temperature control module (not shown in the figure), which is connected with a far infrared heating module for regulating the temperature of the contact place with the human body by raising the temperature of metal electrodes closely attached to skin of the lower limb to provide a hot compress effect. A first light display module 306 is connected to one side of the first wearing portion body 303, and is configured to display the working state of the electrodes, a first pulse control module (a microprocessor) is disposed inside the first wearing portion body 303, and uses an arbitrary waveform generator CPLD (such as a MAX II series chip from Altera), a first pulse communication module and a first pulse power supply module, wherein the first pulse control module is a control circuit board, the first pulse communication module is a Bluetooth module, the first pulse communication module is in signal connection with the main control communication module, and after receiving instructions from the main control unit 1, the first pulse control module controls the first pulse stimulation electrodes 301 and the third pulse stimulation electrodes 302 to emit electric pulse signals according to a specific modulation wave, frequency, bandwidth, amplitude, sequential interval, and spatial position. The first pulse power supply module is a rechargeable lithium battery, and in order to provide electrical energy to the rechargeable lithium battery, a first pulse charging interface 307 is connected to the first wearing portion body 303. The first pulse control module is also connected with a first pulse display module 308, which is configured to display information such as the electric quantity, temperature, operation time of the first electrical pulse assembly 3.

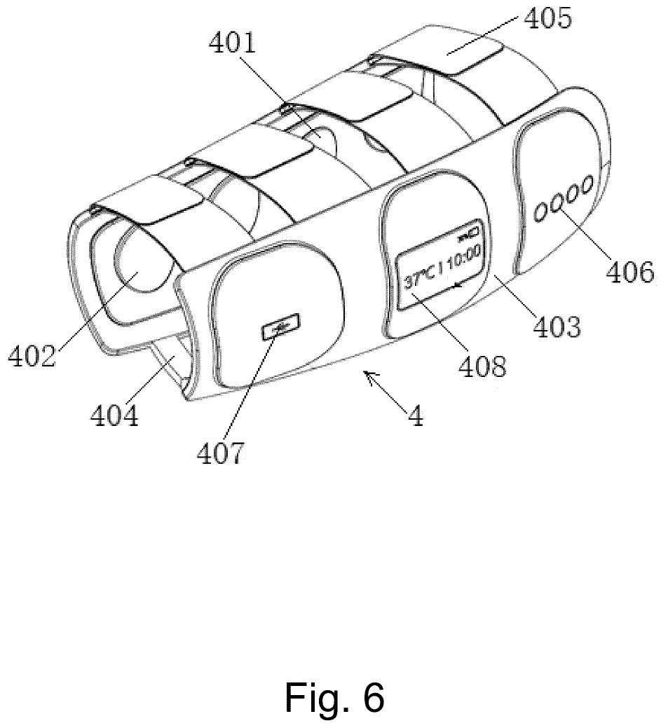

In the first electrical pulse assembly 3 provided in this embodiment, the rechargeable battery, the control circuit board, the Bluetooth module, etc. are packaged in the first wearing portion body 303 made of plastic, thereby improving the integration degree and reducing the size of the product. In addition, the first wearing portion body 303 has a concave circular arc surface, and is better adhered to skin of the thigh of the human body, so that the first pulse stimulation electrodes 301 and the third pulse stimulation electrodes 302 are better adhered to the skin of the thigh, the first wearing portion body 303 specifically includes two plastic sheet-like structures corresponding to the outer thigh and the inner thigh, respectively, the two plastic sheet-like structures are connected by the first hinged connectors 304 and the adjustable elastic bands 305, and in this embodiment, four first hinged connectors 304 and four adjustable elastic bands 305 are provided, and connected to the first wearing portion body 303 at intervals. This adjustable connection structure makes the first electrical pulse assembly 3 easy to wear, conform to ergonomics, tighter to wear, and more comfortable. The first hinged connectors 304 and the adjustable elastic bands 305 make the adjustment of the first wearing portion body 303 more flexible and precise, and can also reduce the probability of displacement of the pulsed stimulation electrodes caused by mechanical vibrations during intervention. The control circuit board integrates a booster circuit, a rectifying circuit, a filter circuit, and a voltage stabilizing circuit, the booster circuit needs to be designed to raise the voltage of the electronic pulses output to a sufficiently high level since the magnitude of the current output from the electronic pulses is low and the resistance of the body surface of the human body is high. In the patent, a transformer is provided to apply output DC signals to a high-frequency carrier, and the voltage is boosted by the transformer. Further, through the rectifying circuit, the filter circuit and the voltage stabilizing circuit, the electric pulses of a constant current source, which meets the requirements of voltage and current, is finally output to be applied to the human body. The structure of the second electrical pulse assembly 4 is substantially the same as that of the first electrical pulse assembly 3, as shown in FIG. 6, the second electrical pulse assembly 4 includes a second wearing portion, the second wearing portion specifically includes a second wearing portion body 403, the second wearing portion body 403 includes two plastic sheet-like structures having a circular arc-shaped curved surface which are oppositely disposed, the two sheet-like structures are adapted to be adhered to the medial side and the lateral side of the lower leg of the human body, the second wearing portion body 403 is connected with at least one second adjustable connecting part, a wearing space is enclosed by the second adjustable connecting part and the second wearing portion body 403, and by adjusting the second adjustable connecting part, an inner wall surface of the second wearing portion body 403 is closely adhered to the lower leg, wherein the two groups of the second pulse stimulation electrodes 401 and the two fourth pulse stimulation electrodes 402 are respectively attached to an inner side wall of the second wearing portion body 403, and arranged at intervals along the length direction of the second wearing portion body 403, and the second pulse stimulation electrodes 401 and the fourth pulse stimulation electrodes 402 are each disc-shaped electrodes. The second adjustable connecting part includes second hinged connectors 404 connected to one end of the second wearing portion body 401, and second adjustable elastic bands 405 connected to the other end of the second wearing portion body 403, specifically, the second hinged connectors 404 are connected to side edges of the two sheet-like structures, respectively, connecting the two sheet-like structures, and the other ends of the two sheet-like structures are separately connected with the second adjustable elastic bands 405, and the second adjustable elastic bands 405 can be further adjusted for tightness by a hook and loop fastener structure.

Further, the second wearing portion body 403 is connected with a second temperature control module, which is connected with a far infrared heating module for regulating the temperature of the contact place with the human body by raising the temperature of metal electrodes closely attached to the skin of the lower limb to provide a hot compress effect. A second light display module 406 is connected to one side of the second wearing portion body 403, and is configured to display the working state of the electrodes. The second wearing portion body 403 is internally provided with a second pulse control module, a second pulse communication module and a second pulse power supply module, wherein the second pulse control module is a control circuit board, the second pulse communication module is a Bluetooth module, and the second pulse communication module is in signal connection with the main control communication module. The second pulse power supply module is a rechargeable lithium battery, and in order to provide electrical energy to the rechargeable lithium battery, the second wearing portion body 403 is also connected with a second pulse charging interface 407. The second pulse control module is also connected with a second pulse display module 407, which is configured to display information such as the electric quantity, temperature, treatment time of the second electrical pulse assembly 4.

Since the structure and function of the second electrical pulse assembly 4 are substantially the same as those of the first electrical pulse assembly 3, its technical effects and advantages are also substantially the same as those of the first electrical pulse assembly 3, which will not be repeated here.

### Embodiment 2

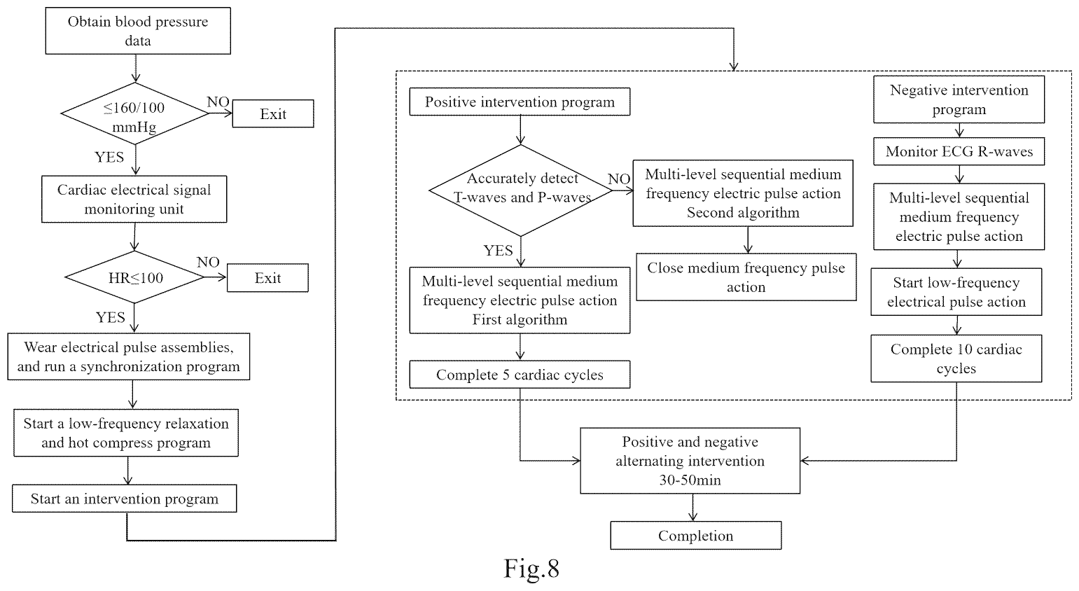

This embodiment provides a control method for the wearable device for alleviating lower limb ischemia provided by Embodiment 1, as shown in FIG. 8, including the following steps:
a blood pressure of the human body is first measured, and if the blood pressure is less than or equal to 160/100 mmHg, procedure control steps are performed:
S1, human body signals are obtained, wherein the human body signals include at least electrocardiosignals.

The signal collecting electrode in the electrocardiosignal monitoring unit 2 measures the electrocardiosignals of the human body.
S2, the obtained human body signals are processed, and control signals are generated according to a processing result including R-wave signals, T-wave signals, or P-wave signals in the obtained electrocardiosignals.

The step S2 specifically includes:
S21, a human body signal processing step, the electrocardiosignals are amplified by the amplifying module, the amplified electrocardiosignals are transmitted to the main control unit 1 by wireless transmission, are filtered in the main control unit 1 and are subjected to A/D conversion by the A/D conversion module, and a cardiac cycle is calculated by the data analysis and processing module, and the R-wave signals, the T-wave signals or the P-wave signals in the electrocardiosignals are obtained.
S22, A determination step, the main control unit 1 determines whether a heart rate (HR) in the electrocardiosignals is less than or equal to 100, and if not, the treatment operation is exited, and if yes, the control signals are generated according to the processing result to synchronize the first electrical pulse assembly 3 and the second electrical pulse assembly 4 with the main control unit 1.
S23, a preheating step, when the HR is less than or equal to 100, the main control unit 1 controls the third pulse stimulation electrodes 302 in the first electrical pulse assembly 3 and the fourth pulse stimulation electrodes 402 in the second electrical pulse assembly 4 to output specific modulation waves with an exponential waveform under the following parameters: a pulse frequency of 50-300 Hz (low frequency); a pulse width of 100-300 μs; a maximum output energy of a single pulse being 250 mJ or less; a single pulse power at a maximum output amplitude of 6 μC or more; and a maximum output amplitude effective value of 25 V or less (50 mA), while the main control unit 1 controls the first temperature control module 305 and the second temperature control module 405 to be heated to 35-45° C., and the time of the preheating step is 3-5 min.

In the preheating step described above, the distal ends of the thigh and the lower leg of the human body are stimulated with low-frequency stimulation pulses, supplemented with heating, so that the effects of relaxing lower limb muscles and blood vessels, reducing vascular impedance, and improving the intervention efficiency of hemodynamics are achieved.
S3, The first electrical pulse assembly and the second electrical pulse assembly are controlled to output pulse modulated waves based on the control signals to alternately perform positive (a return-to-heart direction) stimulation and negative stimulation.

Specifically, when T-waves and P-waves are accurately detected, the second pulse stimulation electrodes 401 are controlled to sequentially output pulse modulated waves in a direction from a distal end of the lower leg to a proximal end of the lower leg, the time interval at which each of the second pulse stimulation electrodes outputs a pulse modulated wave is 10-15 ms, then after the interval of 10-15 ms, the first pulse stimulation electrodes 301 are controlled to sequentially output pulse modulated waves in a direction from a distal end of the thigh to a proximal end of the thigh, each first pulse stimulation electrode 301 is sequentially activated for a time interval of 10-15 ms, and when the P-waves are detected, the first pulse stimulation electrodes 301 and the second pulse stimulation electrodes 401 stop working.

The pulse modulation waves output by the first pulse stimulation electrodes 301 and the second pulse stimulation electrodes 401 are medium-frequency square wave electric pulse modulated waves based on low-frequency modulation, the parameters include a frequency of 1-100 kHz, a pulse width of 200-400 μs, a maximum output amplitude effective value of 25 V or less (50 mA), and a maximum output energy of a single pulse being 300 mJ or less, and the electrical pulse modulated waves can simulate the hammer squeezing effect, and act on the blood vessels of the lower limbs to produce regular deformation and promote blood of the lower limbs to flow back to the upper body during diastole.

When R-waves are detected, the first pulse stimulation electrodes 301 are controlled to sequentially (successively) output medium-frequency square wave pulse modulated waves in a direction from the proximal end of the thigh to the distal end of the thigh; the second pulse stimulation electrodes 401 are controlled to sequentially output pulse modulated waves in a direction from the proximal end of the lower leg to the distal end of the lower leg; and until T-waves are monitored, the first pulse stimulation electrodes 301 and the second pulse stimulation electrodes 302 are controlled to stop working.

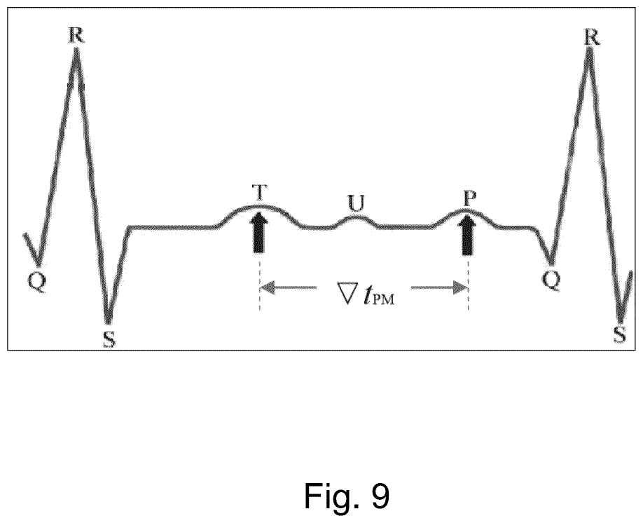

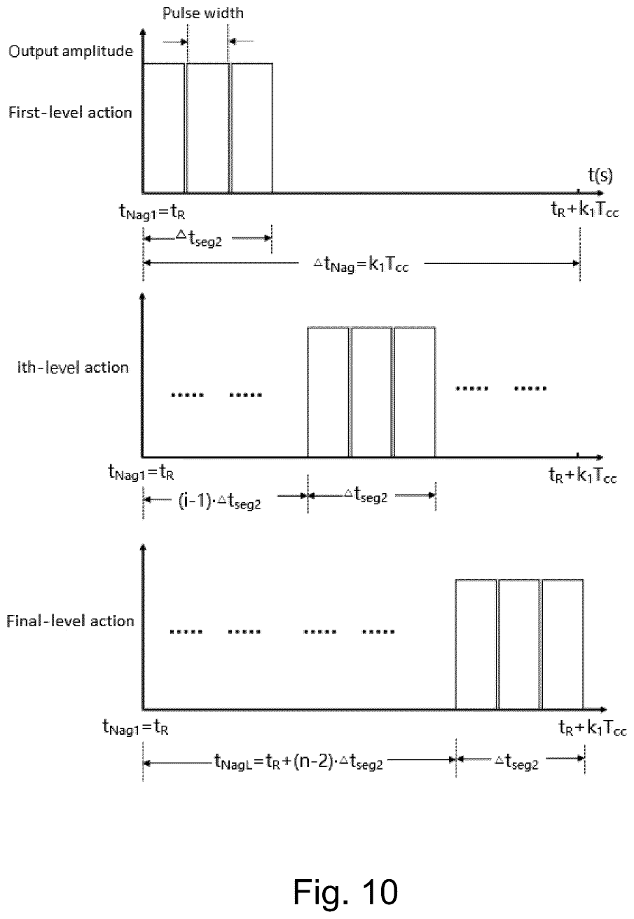

The specific steps and algorithms employed in the above positive stimulation process are as follows:
(1) when the main control unit can accurately obtain T-waves and P-waves in the electrocardiosignals, a first algorithm is employed:
S31, a pressure maintaining duration is calculated based on the detected T-wave signals and P-wave signals in the electrocardiosignals:

$$Δ {t}_{PM} = {t}_{P} - {t}_{T} ;$$

where t_{P} is a time node of a P-wave in the cardiac cycle, and t_{T} is a time node of a T-wave in the cardiac cycle (as shown in FIG. 9).
S32, The main control unit 1 controls the second pulse stimulation electrode 401 (a first level) at the most distal end to be activated to output the medium-frequency square wave electric pulse modulated waves based on the low-frequency modulation as described above (as shown in FIG. 10), and a time node at which the second pulse stimulation electrodes 401 at the most distal end start to be activated: t_{inf l1}=t_{T};
where t_{T} is a time node of a T wave in the cardiac cycle, and a duration of the low-frequency modulation is:

$$Δ {t}_{1} = Δ {t}_{PM} .$$

S33, The main control unit 1 controls the remaining second pulse stimulation electrodes 401 and the first pulse stimulation electrodes 301 (except the first pulse stimulation electrode 301 at the most proximal end) to sequentially (successively) initiate medium-frequency stimulation pulses in a direction from the distal end to the proximal end, forming a multi-level electrical pulse sequential action, and two adjacent levels of pulse stimulation electrodes are activated at a time interval of:

$$Δ {t}_{seg} = \frac{Δ {t}_{infl}}{n - 1} ;$$

where Δt_{inf1} is a duration of the multi-level sequential action, and in this embodiment, the duration is in the adjustable range of 60-100 ms, and n is the number of stages of electric pulse pressurization, n≥3.

In multi-level electrical pulse stimulation, a time node of action of an intermediate-frequency electrical pulse of an ith level (any one of the multi-level electrical pulses):

$${t}_{infli} = {t}_{infl 1} + ( i - 1 ) \cdot Δ {t}_{seg} ;$$

a duration of low-frequency modulation of the intermediate-frequency electrical pulse of the ith level:

$$Δ {t}_{i} = Δ {t}_{PM} - ( i - 1 ) Δ {t}_{seg} .$$

S34, the first pulse stimulation electrode 301 at the most proximal end (a last level) is controlled to initiate a medium-frequency stimulation pulse.

Wherein a time node of action of the first pulse stimulation electrode at the most proximal end is:

$${t}_{inflL} = {t}_{infl 1} + ( n - 2 ) \cdot Δ {t}_{seg} ;$$

a duration of low-frequency modulation of the medium-frequency stimulation pulse is:

$$Δ {t}_{n} = Δ {t}_{PM} - ( n - 2 ) Δ {t}_{seg} .$$

(2) When the main control unit cannot accurately obtain ECG T-waves and P-waves, a second algorithm is employed:
S31′, ECG R-waves are obtained, and the cardiac cycle T_{CC} is calculated and determined.
S32′, The main control unit 1 controls the second pulse stimulation electrode 401 (a first level) at the most distal end to be activated to output the medium-frequency square wave electric pulse modulated waves based on the low-frequency modulation described above, wherein a time node at which the second pulse stimulation electrode 401 at the most distal end starts to be activated: t_{inf l1}=t_{R}+k₁·T_{cc};
where t_{R} is a time node of the R-wave, and k₁ is a constant.

A time node at which the second pulse stimulation electrode 401 at the most distal end is closed: t_{det1}=t_{R}+k₂·T_{cc}.

Where k₂ is a constant, and k₁ and k₂ are in the range of: k₁∈[0.2, 0.25]; k₂∈[0.8,0.85].
S33′, The main control unit 1 controls the remaining second pulse stimulation electrodes 401 and the first pulse stimulation electrodes 301 (except the first pulse stimulation electrode 301 at the most proximal end) to sequentially (successively) initiate medium-frequency stimulation pulses in a direction from the distal end to the proximal end, forming a multi-level electrical pulse sequential action, and two adjacent levels of pulse stimulation electrodes are activated at a time interval of:

$$Δ {t}_{seg 1} = \frac{Δ {t}_{infl}}{n - 1} ;$$

where Δt_{inf1} is a duration of the multi-level sequential action, in the adjustable range of 60-100 ms; and n is the number of stages of electrical pulse pressurization, n≥3.

In multi-level electrical pulse stimulation, a time node of action of an intermediate-frequency electrical pulse of an ith level (any one of the multi-level electrical pulses):

$${t}_{infli} = {t}_{infl 1} + ( i - 1 ) \cdot Δ {t}_{seg 1} ;$$

a duration of low-frequency modulation of the electrical pulse of the ith level:

$$Δ {t}_{i} = Δ {t}_{PM} - ( i - 1 ) \cdot Δ {t}_{seg 1} .$$

S34′, The first pulse stimulation electrode 301 at the most proximal end (a last level) is controlled to initiate a medium-frequency stimulation pulse.

Wherein a time node of action of the first pulse stimulation electrode at the most proximal end is:

$${t}_{inflL} = {t}_{infl 1} + ( n - 2 ) \cdot Δ {t}_{seg 1} ;$$

a duration of low-frequency modulation of the medium-frequency stimulation pulse is:

$$Δ {t}_{n} = Δ {t}_{PM} - ( n - 2 ) \cdot Δ {t}_{seg 1} .$$

The specific steps and an algorithm employed in the above negative stimulation process are as follows:
S35, ECG R-waves are obtained, and a duration of negative stimulation intervention in one cardiac cycle is calculated:

$$Δ {t}_{Nag} = {k}_{1} {T}_{cc} .$$

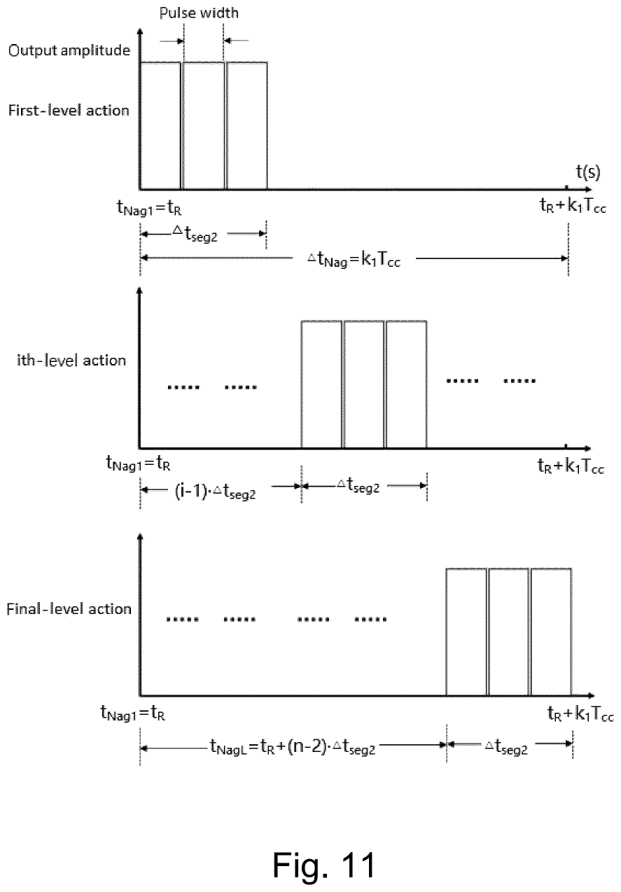

S36, The main control unit 1 controls the first pulse stimulation electrode 301 (a first level) at the most proximal end to be activated to output the medium-frequency square wave electric pulse modulated waves based on the low-frequency modulation described above (as shown in FIG. 11), wherein a time node at which the first pulse stimulation electrode 301 at the most proximal end starts to be activated: t_{Nag1}=t_{R}.
S37, The main control unit 1 controls the remaining first pulse stimulation electrodes 301 and the second pulse stimulation electrodes 301 (except the second pulse stimulation electrode 301 at the most distal end) to sequentially (successively) initiate medium-frequency stimulation pulses in a direction from the distal end to the proximal end, forming a multi-level electrical pulse sequential action, and two adjacent levels of pulse stimulation electrodes are activated at a time interval of:

$$Δ {t}_{seg 2} = \frac{Δ {t}_{Nag}}{n - 1} ;$$

in multi-level electrical pulse stimulation, a time node of action of an intermediate-frequency electrical pulse of an ith level (any one of the multi-level electrical pulses):

$${t}_{Nagi} = {t}_{R} + ( i - 1 ) \cdot Δ {t}_{seg 2} .$$

S34′, The second pulse stimulation electrode 401 at the most distal end (last stage) is controlled to initiate an intermediate-frequency stimulation pulse.

A time node of action of the second pulse stimulation electrode at the most distal end is:

$${t}_{NagL} = {t}_{R} + ( n - 2 ) \cdot Δ {t}_{seg 2} .$$

During the negative stimulation process, the low-frequency electric pulses of the third pulse stimulation electrodes 302 and the fourth pulse stimulation electrodes 402 are continuously activated simultaneously to reduce the vascular impedance at the distal ends of the thigh and the lower leg and direct a blood flow to flow to the distal end.

In this embodiment, controlling the first electrical pulse assembly 3 and the second electrical pulse assembly 4 to output pulse modulated waves to alternately perform positive stimulation and negative stimulation specifically includes: the positive stimulation steps are repeated for 5 cardiac cycles to prompt blood to flow back to the aorta of the upper body, and the negative stimulation steps are repeated for 10 cardiac cycles to prompt blood to flow to the distal ends of the lower limbs, and the positive stimulation and the negative stimulation are repeated for 30-45 minutes to complete the control process of the wearable device.

In this embodiment, by sequentially controlling the first electrical pulse assembly 3 and the second electrical pulse assembly 4, and based on a specific control algorithm, the effect of effectively regulating and controlling the local blood movement of the lower limbs can be achieved, and the technical problems existing in the traditional air pressure driving technology can be solved.

Obviously, the above embodiments are merely examples for clarity of illustration and are not intended to limit the embodiments. Other changes or variations in different forms can be made by those of ordinary skill in the art based on the above description. It is not necessary and impossible to exhaustively list all embodiments here. Obvious changes or variations derived therefrom are still within the scope of protection of the present disclosure.

1. A wearable device for alleviating lower limb ischemia, comprising:
a main control unit,
an electrocardiosignal monitoring unit configured to be worn on a chest of a human body, and being in signal connection with the main control unit;
a first electrical pulse assembly configured to be worn on a thigh of the human body, and being in signal connection with the main control unit, wherein the first electrical pulse assembly comprises at least two groups of first pulse stimulation electrodes, each group of the first pulse stimulation electrodes comprises at least three first pulse stimulation electrodes, the at least two groups of the first pulse stimulation electrodes are respectively disposed corresponding to an outer thigh and an inner thigh, and one ends, away from the electrocardiosignal monitoring unit, of the first pulse stimulation electrodes are provided with at least one group of third pulse stimulation electrodes; and
a second electrical pulse assembly configured to be worn on a lower leg of the human body, and being in signal connection with the main control unit, wherein the second electrical pulse assembly comprises at least two groups of second pulse stimulation electrodes, each group of the second pulse stimulation electrodes comprises at least three second pulse stimulation electrodes, the at least two groups of the second pulse stimulation electrodes are respectively disposed corresponding to a lateral side and a medial side of the lower leg, and one ends, away from the electrocardiosignal monitoring unit, of the second pulse stimulation electrodes are provided with at least one group of fourth pulse stimulation electrodes, wherein
the first pulse stimulation electrodes and the second pulse stimulation electrodes have a pulse frequency of 1-100 KHz, and the third pulse stimulation electrodes and the fourth pulse stimulation electrodes have a pulse frequency of 50-300 Hz.

2. The wearable device for alleviating lower limb ischemia according to claim 1, wherein the first electrical pulse assembly further comprises a first wearing portion, the first wearing portion comprises a first wearing portion body, the first wearing portion body is connected with at least one first adjustable connecting part, a wearing space is enclosed by the first wearing portion body and the first adjustable connecting part, and the first pulse stimulation electrodes and the third pulse stimulation electrodes are attached to an inner wall of the first wearing portion body; and the first wearing portion body is also connected with a first temperature control module.

3. The wearable device for alleviating lower limb ischemia according to claim 2, wherein the second electrical pulse assembly further comprises a second wearing portion, the second wearing portion comprises a second wearing portion body, the second wearing portion body is connected with at least one second adjustable connecting part, a wearing space is enclosed by the second wearing portion body and the second adjustable connecting part, and the second pulse stimulation electrodes and the fourth pulse stimulation electrodes are attached to an inner wall of the second wearing portion body; and the second wearing portion body is also connected with a second temperature control module.

4. The wearable device for alleviating lower limb ischemia according to claim 1, wherein the main control unit comprises an adjustable wristband and a main controller connected to the adjustable wristband, and the main controller comprises a microprocessor, and a communication module, a data analysis and processing module, a data storage module, a display module and a control module which are in signal connection with the microprocessor.

5. The wearable device for alleviating lower limb ischemia according to claim 4, wherein the electrocardiosignal monitoring unit comprises an adjustable chest strap and an electrocardiosignal acquisition module connected to the adjustable chest strap, the electrocardiosignal acquisition module being wirelessly connected with the communication module.

6. The wearable device for alleviating lower limb ischemia according to claim 3, wherein the first adjustable connecting part comprises first hinged connectors connected to one end of the first wearing portion body, and first adjustable elastic bands connected to the other end of the first wearing portion body; and the second adjustable connecting part comprises second hinged connectors connected to one end of the second wearing portion body, and second adjustable elastic bands connected to the other end of the second wearing portion body.

7. A control method for the wearable device for alleviating lower limb ischemia according to claim 1, comprising the steps of:
obtaining human body signals, the human body signals comprising at least electrocardiosignals;
processing the human body signals, and generating control signals according to a processing result comprising R-wave signals, T-wave signals, or P-wave signals in the obtained electrocardiosignals; and
controlling the first electrical pulse assembly and the second electrical pulse assembly to output pulse modulated waves based on the control signals to alternately perform positive stimulation and negative stimulation.

8. The control method according to claim 7, wherein controlling the first electrical pulse assembly and the second electrical pulse assembly to output pulse modulated waves based on the control signals for positive stimulation comprises:
controlling the second pulse stimulation electrodes to sequentially output pulse modulated waves in a direction from a distal end of a lower leg to a proximal end of the lower leg; and
controlling the first pulse stimulation electrodes to sequentially output pulse modulated waves in a direction from a distal end of a thigh to a proximal end of the thigh after a preset time interval.

9. The control method according to claim 8, wherein controlling the first electrical pulse assembly and the second electrical pulse assembly to output pulse modulated waves based on the control signals for negative stimulation comprises:
controlling the first pulse stimulation electrodes to sequentially output pulse modulated waves in a direction from the proximal end of the thigh to the distal end of the thigh;
controlling the second pulse stimulation electrodes to sequentially output pulse modulated waves in a direction from the proximal end of the lower leg to the distal end of the lower leg; and
until T-waves are monitored, controlling the first pulse stimulation electrodes and the second pulse stimulation electrodes to stop working.

10. The control method according to claim 9, wherein the negative stimulation further comprises a step of controlling the third pulse stimulation electrodes and the fourth pulse stimulation electrodes to output pulse modulated waves; and
the method further comprises the steps of controlling the third pulse stimulation electrodes and the fourth pulse stimulation electrodes to output pulse modulated waves, and controlling the first electrical pulse assembly and the second electrical pulse assembly to be heated to 35-45° C. before controlling the first electrical pulse assembly and the second electrical pulse assembly to output pulse modulated waves based on the control signals to alternately perform positive stimulation and negative stimulation.
